(19) United States
(12) Patent Application Publication
(10) Pub. No.: US 2024/0334941 A1
(43) Pub. Date: Oct. 10, 2024

(54) HIGH-EFFICACY NATURAL PRESERVATIVES

(71) Applicant: BioVeritas, LLC, Bryan, TX (US)

(72) Inventors: Cesar B. Granda, College Station, TX (US); Jubo Zhang, College Station, TX (US)

(21) Appl. No.: 18/624,398

(22) Filed: Apr. 2, 2024

Related U.S. Application Data
(60) Provisional application No. 63/457,235, filed on Apr. 5, 2023.
(60) Provisional application No. 63/511,840, filed on Jul. 3, 2023.
(60) Provisional application No. 63/619,899, filed on Jan. 11, 2024.

(51) Int. Cl. A21D 2/14; A21D 15/00; A23L 3/3499; C12P 7/40

(52) U.S. Cl. CPC A21D 2/145; A21D 15/00; A23L 3/3499; C12P 7/40

(57) ABSTRACT

A method for producing a preservative salt includes feeding a biodegradable feedstock to a mixed consortium of microorganisms or microbiomes to produce a fermentation effluent comprising a carboxylic acid mixture; recovering the carboxylic acid mixture from the fermentation effluent; separating an acetic acid and propionic acid fraction from the carboxylic acid mixture; and reacting the acetic acid and the propionic acid fraction with a base to produce the preservative salt comprising an acetic acid and propionic acid salt. The carboxylic acids have greater than 80% bio-based carbon content.

### CROSS-REFERENCE TO RELATED APPLICATIONS

This application claims the benefit of priority to U.S. Provisional Application Nos. 63/457,235 filed Apr. 5, 2023; 63/511,840 filed Jul. 3, 2023; and 63/619,899 filed Jan. 11, 2024, all of which are herein incorporated by reference in their entirety for any and all purposes.

### FIELD

The present invention relates to producing high efficacy natural preservatives from the fermentation or culturing of a biodegradable feedstock.

### BACKGROUND

Fermentation is a natural process that occurs in many environments. The metabolites from fermentation tend to be innocuous and even beneficial to the host (den Besten et al., 2013). The most common metabolites are organic acids, such as short- and medium-chain carboxylic acids (SCCAs and MCCAs). Among the SCCAs produced by fermentation, acetic acid and propionic acid are used to preserve foodstuffs such as bread, tortillas, meats, and sauces. The calcium salt of propionic acid is commonly used as a mold inhibitor (Stopforth et al., 2005) and has been used as such since the 1930's. It is produced from the neutralization of propionic acid with calcium hydroxide (lime). Propionic acid is typically produced from petroleum, which raises sustainability and contamination concerns. Other natural processes generate propionic acid and its salts, in addition to other organic acids, through fermentation using microorganisms such as Propionibacterium. Such products are considered to be “natural” and/or “clean label.” However, the fermentation effluents are typically cleaned and dried into a powder without much purification. As a result, these natural products tend to be significantly less efficacious than petroleum-derived propionate products, thus requiring higher dosages of the natural product that can, in turn, affect the organoleptic (e.g., aroma, taste, texture, etc.) properties of the foods in which it is used as a preservative (Verheezen et al., 2020).

### SUMMARY

Provided herein is a preservative salt produced from a natural biodegradable feedstock, and a method for producing and using the same. Carboxylic acids such as acetic, propionic, butyric, iso-butyric, valeric, and iso-valeric (carbon chain length ranging from C2 to C5, i.e., short-chain carboxylic acids), and caproic, heptanoic, caprylic, and nonanoic (carbon chain length ranging from C6 to C9, i.e., medium-chain carboxylic acids) can be produced from mixed culture or mixed consortium fermentation followed by acid recovery. The short-chain acids, namely acetic and propionic acid, may be used to manufacture salts, which can be used as natural and clean-label preservatives.

In one aspect, a method is provided for obtaining carboxylic acids from a mixed consortium or microbiome fermentation. The method includes a carboxylic acid recovery step from the mixed consortium or microbiome fermentation by separating the carboxylic acids through distillation or freeze crystallization. The carboxylic acids that are recovered include acetic and propionic acids that are then reacted with, or neutralized with, a base to produce carboxylic acid salts. These carboxylic acid salts may be used as preservative salts. In some embodiments, water is added before or after the neutralization reaction to allow the preservative salts to be fully soluble so as to filter them to remove insoluble impurities. In some embodiments, water is added before the neutralization reaction. The salts may then be dried using a spray dryer, a drum dryer, or the like. The salts may be subjected to crystallization, recovered, and dried. In some embodiments, the salts are first crystallized, separated and then dried using a convection dryer.

Illustrative bases include, but are not limited to, sodium hydroxide, potassium hydroxide, calcium hydroxide, sodium carbonate, potassium carbonate, calcium carbonate, or a mixture of any two or more thereof. In some embodiments, an acetate and/or propionate portion of the total mass of the preservative salt is from about 60 wt. % to 80 wt. % on a dry basis. In other embodiments, it is from about 70 wt. % to 80 wt. % by mass. In some embodiments, the mass fraction of propionate in the acetate and propionate portion of the preservative salt is from about 50 wt. % to 99.9 wt. % by mass, or from about 80 wt. % to 90 wt. % by mass. In some embodiments, the preservative salt includes calcium propionate.

In another aspect, a preservative salt is prepared according to the methods described above. In some embodiments, the preservative salt is a clean label salt. In some embodiments, the preservative salt is naturally sourced. In some embodiments, the preservative salt is sourced from biodegradable materials. In some embodiments, the preservative salt is not petroleum-based. In some embodiments, the preservative salt is substantially free of petroleum products.

In a further aspect, a method of using the preservative salts produced by the disclosed process is provided. In the methods, a preservative salt is added to a dough for a baked commodity. As an illustration, the baked commodity may be bread, a flour tortilla, a corn tortilla, or a bagel. In some embodiments, the baked commodity is bread. The preservative salt may be added to the dough at a weight percentage of about 0.3 wt. % to 1.0 wt. % based on the total dry weight of flour in the dough.

In yet another aspect, a method is provided for producing preservatives from carboxylic acids produced in fermentation. The method includes fermenting a biodegradable feedstock using a mixed consortium of microorganisms to produce a fermentation effluent including carboxylic acids; recovering a carboxylic acid product from the fermentation effluent using a carboxylic acid recovery system; separating one or more fractions from the carboxylic acid product, the one or more fractions comprising at least 90 wt. % of acetic acid, propionic acid, or a combination thereof; and reacting the one or more fractions with a base to produce a preservative salt.

The carboxylic acid product of the method may include carboxylic acids, carboxylic acid salts, or combinations of any two or more thereof. The biodegradable feedstock used in the process may be a byproduct or a waste material from the food industry and/or the agricultural industry. In some embodiments, the byproduct or waste material includes, but is not limited to, by-products or wastes from the food and agricultural industry, such as, dairy sources (including milk, milk solids, whey, whey powder, acid whey, whey permeate, lactose, or yogurt), sugars (including those derived from corn, beet, palm, or sugar cane), sugar sources (including maple syrup, molasses, caramel, or soda syrup), starch and fiber sources (including those from pea, oat, barley, corn, potato, rice, wheat, milo, malt, tapioca, pasta, or bread), fruit- and vegetable-based sources (including juices, pastes, or peels), vegetable-based oils (including canola oil, corn oil, peanut oil, soybean oil, palm oil, rapeseed oil, or their derived glycerol and fatty acids), vegetable-based proteins (including soy protein, pea protein, wheat protein, corn protein, or their concentrates and isolates), other protein and nutrient sources (whey, soybean meal, corn steep liquor, yeast extract).

The separation of one or more carboxylic acid fractions from the carboxylic acid product may be performed using a distillation system. In some embodiments, separation of the one or more fractions from the carboxylic acid product may be performed by freeze crystallization. The carboxylic acid fraction may include acetic acid, propionic acid, or both.

In some embodiments, the base is sodium hydroxide, potassium hydroxide, calcium hydroxide, sodium carbonate, potassium carbonate, calcium carbonate, sodium bicarbonate, potassium bicarbonate, calcium bicarbonate, or combination a combination of any two or more thereof.

In combination with any of the previous embodiments, the method may also include adding water to the one or more carboxylic acid fractions such that the salts are solubilized. In some embodiments, the water is added before reacting the one or more fractions with the base to produce the preservative salt. In some embodiments, the water is added after reacting the one or more fractions with the base to produce the preservative salt. In some embodiments in which water is added, soluble preservative salts are dissolved into a preservative salt solution. In some embodiments, the preservative salt solution is filtered to remove insoluble impurities.

In combination with any of the previous embodiments, the preservative salts may be dried into a powder using a spray dryer or a drum dryer. In some embodiments, the preservative salt is crystallized to form a crystallized preservative salt. The crystallized preservative salt may be recovered, and then dried to produce a powder that contains the preservative salts. In any of the embodiments herein, the powder may be ground and sifted to achieve a desired particle size suitable for ease of handling and low-dust characteristics.

The acetate and propionate may be present in the preservative salts from about 60 wt. % to about 80 wt. % on a dry basis of the total preservative salt. The acetate and propionate may be present in the preservative salts from about 70 wt. % to about 80 wt. % on a dry basis of the total preservative salt. In some embodiments, the propionate in the acetate and propionate portion of the preservative salt is present from about 50 wt. % to 99.9 wt. % on a dry basis. In some embodiments, the propionate in the acetate and propionate portion of the preservative salt is present from about 80 wt. % to 90 wt. % on a dry basis.

In another aspect, a method of using a preservative salt produced according to the methods disclosed above is provided. Such methods include replacing petroleum-based propionate salts as a preservative in various food and feed products with the biodegradable feedstock-based preservative salt disclosed herein. In some embodiments, the preservative salt replaces petroleum-based propionate salts as a natural or clean-label preservative in various food and feed products. In some embodiments, the preservative salts produced using the provided methods have greater than 90% bio-based carbon content as measured by ASTM D6866.

In another aspect, also provided herein is a food product containing a preservative salt prepared according to the methods disclosed herein. The food product may be a baked composition. In some embodiments, the baked composition maintains a degree of difference in sensory testing of less than 1.5 after 9 days of aging when compared to a second baked composition comprising calcium propionate prepared by the reaction of a calcium salt with a petroleum-derived propionic acid. In some embodiments, the baked composition includes flour and the calcium propionate is present from about 0.3 to 1.0 wt. % based on the amount of flour in the baked composition.

### BRIEF DESCRIPTION OF DRAWINGS

FIG. 1 is a data plot representing the number of days before mold was observed on test loaves (pup loaves) treated with a preservative salt as described herein, test loaves (pup loaves) left untreated or treated with comparative preservatives, according to the examples.

FIG. 2 is a data plot representing the number of days before mold was observed on a tortilla treated with a preservative according to the examples, compared to baked commodities left untreated or treated with comparative preservatives.

FIG. 3 is a flowchart illustrating the steps of the method of producing a naturally-source preservative salt, according to various embodiments.

FIG. 4 is a principal component biplot of sensory means of breads treated with various preservative salts produced or procured according to the examples.

FIG. 5 is a principal component biplot of sensory means of breads treated with various preservative salts produced or procured according to the examples.

FIG. 6 is a principal component biplot of sensory means of breads treated with various preservative salts produced or procured according to the examples.

FIG. 7 is a principal component biplot of sensory means of breads treated with various preservative salts produced or procured according to the examples.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As utilized herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values. When “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

It should be understood that, although example implementations of embodiments are described herein, the systems, methods, and uses may be implemented using any number of techniques, whether currently known or not. It is also noted that the “process streams” described herein need not be clean cut or pure. When referring to particular reactant and product streams herein, it should be understood that, although the primary product(s) may be described, other products may exist in the streams. Thus, there may be quantities of the other compounds in such streams and/or other impurities.

The use of mixed organic acids for the preservation in food is not a new concept (Doores, 2005). Anderson (1957) had suggested the use of a mixture of acetic and propionic acid to produce calcium acetate propionate, which on a propionate weight basis, was shown to be more effective in preservation of bread than calcium propionate. Verheczen et al. (2020) in U.S. patent application Ser. No. 16/887,210, and Roozen et al. (2015) in U.S. Pat. No. 11,292,997 proposed formulations where vinegar is buffered and added to propionate prepared by fermentation. The push for sustainably and naturally produced ‘clean-label’ preservatives has resulted in many commercial products (Nachay, 2020), where the propionic, acetic acid and other acids, which may be buffered with a base, come from fermentation (Verheezen et al., 2020). The term ‘clean label’ is defined as products that avoid artificial additives, synthetic chemicals, and other ingredients that consumers might perceive as unhealthy or unfamiliar. Current commercial ‘clean-label’ products may meet the above definition, however they lack the purity of the products made using the methods discussed herein, which lessens their efficacy and their organoleptic properties. In addition, the low efficacy causes the need for the preservative to be dosed in larger quantities, which further exacerbates any negative sensorial attributes.

The present systems and methods may be used to produce high efficacy preservatives, which in turn may be used as high efficacy preservatives in food products. The high efficacy preservatives as provided herein may be considered to be naturally sourced. Accordingly, the high efficacy preservatives include substantial amounts of bio-based carbon and are substantially free of petroleum-based carbon. Use of these high efficacy preservatives in food products is also discussed in more detail below.

The high efficacy preservatives are preferably produced from carboxylic acids produced and recovered from fermentation. Thus, the present methods include a methods to produce carboxylic acids in a fermentation and methods to recover the carboxylic acids from the fermentation products through the use of extracting solvents, followed by distillation. The fermentation utilizes natural biodegradable feedstocks which are described in more detail below. The natural biodegradable feedstocks are substantially free of products, byproducts, or waste products of petroleum refining, which are considered as synthetic or artificial. As the feedstock is a natural biodegradable feedstock, the produced carboxylic acids and preservative salt products are substantially free of petroleum-based carbon. In some embodiments, the carboxylic acids produced using the provided process have greater than 80% bio-based carbon content or greater than 90% bio-based carbon, as measured by ASTM D6866. The distillation of the fermentation products generally provides a mixture of acetic and propionic acid that can then be buffered with a suitable base, followed by drying to provide a salt of the carboxylic acids (such as acetic acid salt and/or propionic acid salt). These carboxylic acid salts can be combined and processed to form the high efficacy preservatives.

### Production and Recovery of Organic Acids

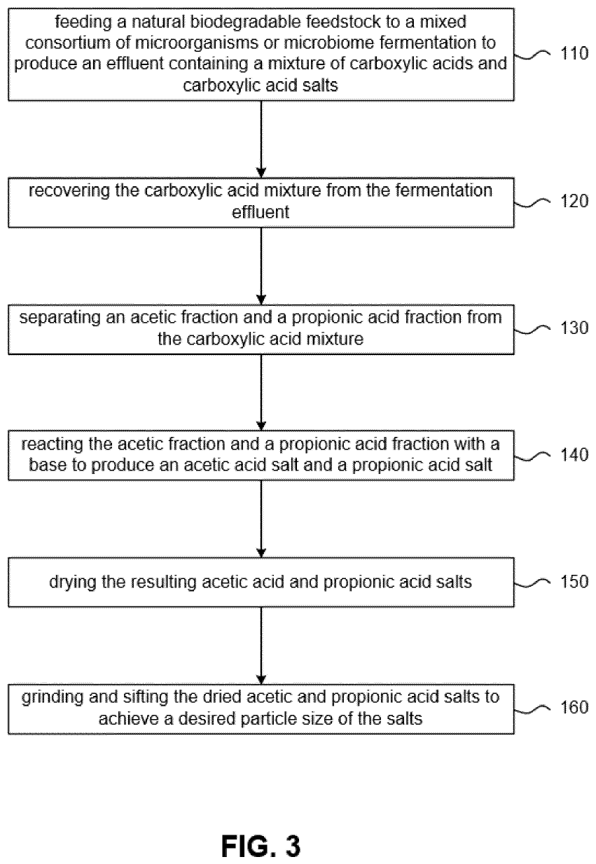

Referring to FIG. 3, a process for producing carboxylic acid salts from a natural biodegradable feedstock for use as a preservative may entail several steps. The produced carboxylic acid salts may include acetic acid salts and propionic acid salts. First, a fermentation step 110 is employed to produce the organic acids with the desired characteristics. In this step, a natural biodegradable feedstock is introduced into a fermentor containing a mixed consortium of microorganisms or a microbiome. A single fermentor may be used, or multiple fermentors arranged in series or in parallel may also be used. A fermentor may be any suitable vessel that allows for the control of the operating conditions as described herein. In some embodiments, the fermentors are continuously stirred tank reactors (CSTRs). In other embodiments, the fermenters are standard batch reactors.

Natural microbiomes are mixed consortia of microorganisms that exist in nature. These microorganisms generally produce a mixture of organic acids, namely carboxylic acids, including short- and medium-chain fatty acids (SCFAs and MCFAs) as metabolites from the fermentation or digestion of biodegradable feedstock. The carboxylic acids may include acetic acid (C2) up to pelargonic acid (C9), including acetic acid (C2), propionic acid (C3), butyric acid (C4), valeric acid (C5), carproic acid (C6), enanthic acid (C7), caprylic acid (C8), and pelargonic acid (C9). These acids may also be known by their IUPAC names: ethanoic acid (C2), propanoic acid (C3), butanoic acid (C4), pentanoic acid (C5), hexanoic acid (C6), heptanoic acid (C7), octanoic acid (C8), and nonanoic acid (C9). Smaller amounts of acids having carbon chains larger than C9 may also be present, but such larger acids would be expected to be at very low amounts.

Through controlled fermentation conditions, the product profile of the carboxylic acid products can be adjusted to be in the short range of C2 and C3 carbon numbers (i.e., acetic and propionic acids) or in the medium range of C4 to C9 carbon numbers (i.e. butyric through pelargonic acids, inclusive) or to include a mixture of acids from both ranges. Such conditions may include feedstock formulation, temperature, pH, loading rate, and liquid residence time.

Feedstocks that may be used include, but are not limited to, by-products or wastes from the food and agricultural industry, such as, dairy sources (including milk, milk solids, whey, whey powder, acid whey, whey permeate, lactose, yogurt), sugars (including those derived from corn, beet, palm, or sugar cane), sugar sources (including maple syrup, molasses, caramel, soda syrup), starches and starch and fiber sources (including those from pea, oat, barley, corn, potato, rice, wheat, milo, malt, tapioca, pasta, bread), fruit- and vegetable-based sources (including juices, pastes, peels), vegetable-based oils (including canola oil, corn oil, peanut oil, soybean oil, palm oil, rapeseed oil and their derived glycerol and fatty acids), vegetable-based proteins (including soy protein, pea protein, wheat protein, corn protein and their concentrates and isolates), other protein and nutrient sources (whey, soybean meal, corn steep liquor, yeast extract). The precise composition of the consortia of microorganisms that participate in the digestion and fermentation reactions will vary based on the feedstock provided and thus so will the organic acid profile of the product stream. Controlling the feedstock can influence which microorganisms flourish within the fermentor system and thus also the acid profile.

Conditions can also influence which microorganisms flourish within the fermentor system and the acid profile. Among those, temperature, pH, organic matter loading rate and liquid residence time. Typical temperatures that are used in mixed-culture or mixed consortium fermentation are from about 35 to 60° C. Colder temperatures will slow the fermentation and digestion processes and may require additional residence time to completely consume the feedstock. Higher temperatures may increase fermentation and digestion rates, but too high of a temperature may stress or even kill the microbiome. Temperature control can influence the microbiome make-up and organic acid profile of the product stream. In some embodiments where short-chain acids production is desired, the fermentor is operated at a temperature from about 35 to about 45° C. to maximize production of short-chain acids.

The pH will also affect the microbial composition of the microbiome and the final profile of the acids produced. However, too low of a pH will inhibit microorganisms. Thus, suitable pH ranges for production of acids range from about 5 to about 9. This may include a pH range from 6 to 8 to favor short-chain acids production. In addition, another control parameter is the organic matter loading rate, expressed as volatile solids loading rate (VSLR), which sets the size of the fermentor or fermentors. Volatile solids are considered to be the ash-free portion of biodegradable feedstock, which is a proxy for organic matter in the feedstock. Another control parameter is hydraulic or liquid residence time (LRT). Longer residence times will select for slower growing microorganisms; shorter residence times will select for faster growing ones. In some embodiments, LRT will vary between a few hours to several days. Natural microbiomes producing organic acids in animal rumens, for instance, can have residence times of up to 72 hours (Weimer et al., 2009). In general, testing of the various conditions for a given culture may be performed to determine the profile of the produced organic acids. The various conditions in a reactor may be adjusted to achieve a desired organic acid profile.

Referring again to FIG. 3, a method of producing clean label carboxylic acid salts from a biodegradable feedstock includes feeding the biodegradable feedstock to a consortium of microorganisms or microbiome for fermentation 110. The fermentation step 110 results in an effluent or fermentation broth that contains a mixture of carboxylic acids and carboxylic acid salts.

With continued reference to FIG. 3, the natural carboxylic acids are recovered from the fermentation product stream (the fermentation effluent or broth) using several clean methods in an acid recovery step 120. For example, in some embodiments, this step is performed at least in part by acidification of the product stream by carbonic acid or CO₂ and extraction, followed by distillation (Ross and Granda U.S. Pat. No. 10,662,447) in a carboxylic acid recovery system (CARS). The fermentation product stream may be clarified or filtered before the recovery step, if desired, such that suspended solids present in the fermentation product stream are substantially removed from the product stream. The recovery step 120 results in a stream including the recovered carboxylic acids at substantially higher concentrations than the concentrations that were present in the fermentation product stream.

### Production of Organic Salts for Preservation

With continued reference to FIG. 3, the recovered carboxylic acids may be separated from the extraction solvent and heavier acids in a separation step 130. This may be carried out by, for example, distillation or in some embodiments by freeze crystallization (Barr and Newsham, 1986). In a distillation process utilizing a distillation column, after lighter constituents like CO₂ and, optionally, water have been removed, acetic and propionic acids can be recovered in one or more carboxylic acid fractions. The acetic and propionic acids may be collected in a single combined carboxylic acid fraction or in separate fractions (i.e., an acetic acid fraction and a propionic acid fraction). Carboxylic acid fractions having different concentrations or mass fractions of acetic and propionic acids can be obtained, which range from 50 wt. % propionic acid component up to 99 wt. % propionic acid component of the sum of the acetic and propionic acid. Further distillation and other separation processes may optionally be used to adjust the carboxylic acids fractions to a desired concentration or composition.

The resulting acetic and propionic acid fractions can be buffered with base in a buffering step 140 in a manner similar to the production of buffered vinegar (Younes et al., 2022), where base, such as but not limited to sodium hydroxide, potassium hydroxide, calcium hydroxide or their carbonates, bicarbonates or combination of any two or more thereof, are used to neutralize the acids to reach a target pH ranging from 6.5 to 8.5, but more precisely from 7 to 8. This buffering step results in a salt solution including soluble acetate and propionate salts. Water may optionally be added to the salt solution before or after the buffering step 140 to ensure the salts are fully soluble before filtering.

Once the desired pH has been reached, the salt solution may be filtered to remove insoluble species and then concentrated and dried in a drying step 150. The drying step may use any suitable mean such as, but not limited to, a spray dryer, or a drum dryer, or by crystallization following by filtration or centrifugation of the crystals and convection drying to produce a dried preservative salt.

In an optional grinding step 160, further grinding and sifting of the resulting dry preservative salt to a desired particle size may also be implemented. The particle size of the preservative salt is a consideration, as too large of a particle size may not allow adequate incorporation of the preservative salt into the food or feed product needing preservation, and too small of a particle size may cause excessing dusting during handling of the preservative salt. For example, in some embodiments, the desired particle size may be such that greater than 95% of the powder passes through a #30 sieve and greater than 90% of the powder is retained by a #100 mess, on a mass basis. In some embodiments, the desired particle size may be such that greater than 95% of the powder passes through a #20 sieve and greater than 90% of the powder is retained by a #80 mess, on a mass basis.

When diluted to an approximately 10 wt. % solution, the resulting preservative salt will have a pH ranging from 7 to 9. The resulting salts of the carboxylic acids will be substantially acetate and propionate and the metal counter ion, such as, but not limited to sodium, potassium, calcium, or a combination of any two or more thereof. The acetate/propionate portion of the total salt weight (carboxylate plus metal cation) may be from about 60 wt. % to 80 wt. % on a dry basis. The mass fraction of propionate in the acetate/propionate portion of the preservative salt may range from about 50 wt. % to 99.9 wt. % or from 80 wt. % to about 90 wt. %.

The preservative salts produced by the methods described herein are substantially free of petroleum-sourced material, and are also free of any potential contaminants associated with petroleum products. Because they employ a natural, biodegradable material source, the disclosed preservative salts may be termed to be “clean label” or “natural,” in contrast to those produced using petroleum-based materials. The lack of petroleum-based material in the preservatives salts can be confirmed by measuring the amount of carbon 14 (¹⁴C) in a sample and determining the amount of carbon in the sample that is “bio-based.” Because bio-based carbon is relatively young compared to what is found in petroleum samples, the amount of ¹⁴C in the preservative salts will be higher than what is found in petroleum salts produced using petroleum-based feedstocks. In some embodiments, the preservative salts produced using the provided methods have greater than 80% bio-based carbon content as measured by ASTM D6866. In some embodiments, the preservative salts produced using the provided methods have greater than 90% bio-based carbon content or even greater than 95% bio-based carbon as measured by ASTM D6866. The preservative salts produced by the provided method may have a higher efficacy than those taught in previous art.

The present invention, thus generally described, will be understood more readily by reference to the following examples, which are provided by way of illustration and are not intended to be limiting of the present invention.

### EXAMPLES

Example 1. This example describes the manufacturing of a natural, clean-label salt preservative. The process was performed under current good manufacturing practices (cGMP). Fermentation from a wheat-based starchy food industry byproduct was carried out using a mixed culture or mixed consortium of microorganisms, which form a stable microbiome, at a pH of 6.8. This fermentation pH was controlled by adding sodium and potassium carbonates and bicarbonates. The temperature of the fermentation was controlled at 40° C. A mixture of salts of carboxylic acids were produced consisting substantially of acetate, propionate, butyrate, isobutyrate, valerate, isovalerate, caproate, heptanoate and octanoate at a total titer of over 30 g/L, where the % of acetate was about 40% and propionate was 15%. The fermentation effluent containing the salts was further cleaned with ultra-filtration membranes and was concentrated through evaporation to then recover the carboxylic acids by extraction. Acetic acid and propionic acid were separated from the rest of the carboxylic acids by distillation. A mixture of recovered natural acetic (13 wt. %) and propionic (87 wt. %) acids were used to prepare a calcium-based clean-label natural preservative by neutralizing to a pH of about 7 the acid mix with the addition a slurry consisting of water and food-grade lime (calcium hydroxide). The resulting acetate/propionate preservative salt solution was filtered to removed insoluble particles and then dried to a powder in a convection oven. The resulting clean-label natural preservative can be employed as a mold inhibitor as a direct replacement for petroleum-based calcium propionate. Table 1 shows the composition of the resulting preservative.

TABLE 1

| | | |
|---|---|---|
| Composition | Method | Values |

Assay

| | | |
|---|---|---|
| Acetate (wt. %) | GC-FID | 10.4 |
| Propionate (wt. %) | GC-FID | 69.6 |

Metals and Fluoride

| | | |
|---|---|---|
| Calcium (wt. %) | ICP-MS | 21.1 |
| Arsenic (ppm) | ICP-MS | 0.072 |
| Cadmium (ppm) | ICP-MS | 0.027 |
| Lead (ppm) | ICP-MS | 0.019 |
| Mercury (ppm) | ICP-MS | <0.005 |
| Iron (ppm) | ICP-MS | 5.7 |
| Magnesium | ICP-MS | 967.4 |
| (ppm) | | |
| Potassium (ppm) | ICP-MS | 15.3 |
| Sodium (ppm) | ICP-MS | 30.6 |
| Fluoride (wt. %) | ISE | <0.001 |
| Moisture (wt. %) | Drying | 1.54 |
| pH (10% | pH meter | 8.4 |
| solution) | | |

Example 2. A comparative shelf-life study was performed using pup loaves by an ISO/IEC 17025:2017 certified laboratory, Great Plains Analytical Laboratory, Kansas City, MO, USA. A standard white bread dough formulation was prepared (pup loaves) for each test group. Approximately 500 grams of dry material was used to prepare 3 pup loaves per test group. Various preservatives, which were to act as mold inhibitors, were added to the doughs. The amount of preservatives added to each dough is reported as a weight percentage based on the mass of flour in the dough (“on flour”). Each mold inhibitor was thoroughly mixed into the formulation for each dough. The percentage inclusion and type of mold inhibitor on flour is outlined in Table 2. Three of the mold inhibitors were made with acetic and propionic acid produced from fermentation using a mixed consortium of naturally occurring microorganisms or microbiome using the methods previously described herein. A first comparative mold inhibitor was a natural calcium propionate (BV CP), which was over 99 wt. % calcium propionate. The second and third comparative mold inhibitors were high-efficacy clean-label acetate/propionate mold inhibitors, with one having an acetate/propionate weight ratio of 1:1 (50 wt. % acetate/50 wt. % propionate) (BV1) and the other one having an acetate/propionate weight ratio of 1:6 (14 wt. % acetate/86 wt. % propionate) (BV2). These inventive mold inhibitors were compared to a leading commercially available clean-label mold inhibitor (CCMI) and to commercially-available petroleum-based calcium propionate (CCP). After mixing, the dough was divided into disposable bread pans and allowed to ferment for 3 hours at 86±1° F. and 60% relative humidity, proofed for 55 minutes at 109±1° F. and 90% humidity, and baked for 15 minutes at 425±1° F. This procedure was repeated for each test group for a total of 30 loaves of bread. The internal temperature of each loaf was recorded upon exiting the oven and prior to packaging (Table 3). In addition, water activity and pH were measured in one of the replicates for each group prior to baking using a water activity meter and a pH meter (Table 4). The product odor (similar to baseline or off odor development), color, and general appearance were evaluated daily. Each sample was opened and closed daily until visible mold appeared.

TABLE 2

| | Sample | wt. % on |
|---|---|---|
| Preservative salts acting as mold inhibitor | ID | flour |
| Control (no additive) | Control | 0 |
| Commercial Petroleum-based calcium | CCP | 0.3 |
| propionate | | |
| High-efficacy clean-label acetate/propionate 1:1 | BV1 | 1 |
| High-efficacy clean-label acetate/propionate 1:1 | BV1 | 0.3 |
| High-efficacy clean-label acetate/propionate 1:6 | BV2 | 1 |
| High-efficacy clean-label acetate/propionate 1:6 | BV2 | 0.3 |
| Leading commercial clean-label mold inhibitor | CCMI | 1 |
| Leading commercial clean-label mold inhibitor | CCMI | 0.3 |
| Natural calcium propionate | BV CP | 1 |
| Natural calcium propionate | BV CP | 0.3 |

TABLE 3

| Sample | wt. % Mold | Bread Temp exiting | Bread Temp prior to |
|---|---|---|---|
| ID | Inhibitor | oven ° F. | packaging ° F. |
| Control | 0 | 202.1 | 67.4 |
| CCP | 0.3 | 202.4 | 67.5 |
| BV1 | 1 | 202.6 | 67.9 |
| BV1 | 0.3 | 202.6 | 67.0 |
| BV2 | 1 | 201.6 | 68.2 |
| BV2 | 0.3 | 200.0 | 68.1 |
| CCMI | 1 | 203.1 | 67.2 |
| CCMI | 0.3 | 201.4 | 67.6 |
| BV CP | 1 | 202.7 | 69.5 |
| BV CP | 0.3 | 202.2 | 68.4 |

TABLE 4

| | Water | |
|---|---|---|
| Sample ID | Activity | pH |
| Control | 0.954 | 5.05 |
| CCP | 0.9564 | 5.40 |
| BV1 | 0.9572 | 5.43 |
| BV1 | 0.9605 | 5.32 |
| BV2 | 0.9516 | 5.51 |
| BV2 | 0.9525 | 5.56 |
| CCMI | 0.9601 | 5.38 |
| CCMI | 0.9544 | 5.36 |
| BV CP | 0.9448 | 5.73 |
| BV CP | 0.9535 | 5.65 |

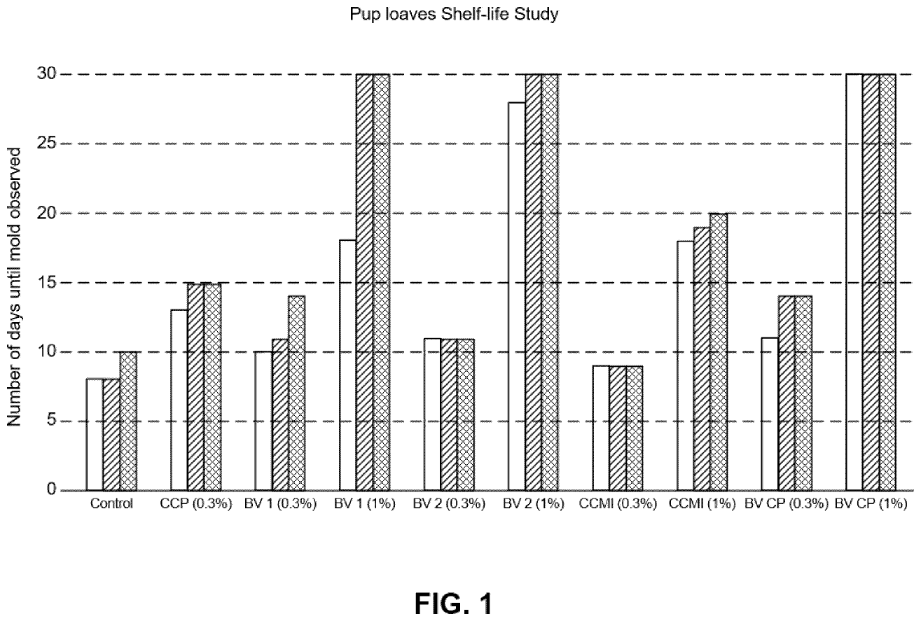

The results for the bread shelf-life study comparing the preservatives/mold inhibitor treatments are shown in FIG. 1. Three sample loaves were produced using each of the inhibitor treatments, and the number of days until mold was observed was recorded for each loaf. The samples were stored from 70° F. to 73° F. (ambient temperature) over the course of the study. The dough pH results were from 5.05 to 5.73. The water activities for the dough were between 0.9448 and 0.9605. The results are presented in Table 4. After baking, it was observed that the treatment with the high-efficacy clean-label acetate/propionate 1:6 (BV2) at 1 wt. % and natural calcium propionate (BV CP) also at 1 wt. % had shorter loaf heights compared to the other samples and had a tighter grain with firmer texture. This is expected due to the high efficacy and the high inclusion rate, which could have inhibited the yeast. Natural calcium propionate (BV CP) at 1 wt. % was the only sample to have all three replicates remained free of visible mold throughout the 30-day study. The treatment with high-efficacy clean-label acetate/propionate 1:6 (BV2) at 1 wt. % had two replicates remain free of visible mold but one replicate developed mold at day 28. The treatment with high-efficacy clean-label acetate/propionate 1:1 (BV1) at 1 wt. % had two replicates remain free of visible mold but one replicate developed mold at day 18. After day 30, the samples were stored under similar conditions, but the bags were no longer opened each day. On day 45, the treatment with high-efficacy clean-label acetate/propionate 1:6 (BV2) at 1 wt. % had one additional replicate develop mold. The two replicates with high-efficacy clean-label acetate/propionate 1:1 (BV1) at 1 wt. % remaining and all replicates of treatment with natural calcium propionate (BV CP) at 1 wt. % showed no signs of mold growth or off odor at day 45. In comparison to the control (no additive), most mold inhibitor treatments performed better, with the leading commercially available mold inhibitor (CCMI) performing the worst at both 0.3 wt. % and at the 1 wt. % inclusion level. This was expected, as present commercially available clean-label mold inhibitors do not display the high efficacy and higher inclusion percentages are needed to achieve the same inhibitor performance as petroleum-based preservatives. As expected, the commercially available petroleum-based calcium propionate (CCP) performed similar to the natural calcium propionate (BV CP) at the 0.3 wt. % inclusion level. Also, at both 0.3 wt. % and 1 wt. % inclusion level, the higher fraction of propionate in the calcium propionates (CCP and BV CP) allowed for better performance with the natural calcium propionate (BV CP) at 1 wt. % holding beyond 30 days. Interestingly, the performance at 0.3 wt. % and at 1 wt. % was very close for the high-efficacy clean-label mold inhibitors at an acetate/propionate ratio of 1:1 (BV1) and 1:6 (BV2). No off-odors were detected over time in any of the treatments.

Example 3. A comparative shelf-life study using flour tortillas was performed by an ISO/IEC 17025:2017 certified laboratory, Great Plains Analytical Laboratory, Kansas City, MO. The purpose of this study was to compare the efficacy of preservatives, which were to act as mold inhibitors, in a tortilla formulation. A total of 10 test groups of tortillas were prepared: 9 test groups contained various mold inhibitors and one test group served as control, without a mold inhibitor (no additive). Test groups were stored at ambient temperature for 30 days to determine the overall efficacy of the inhibitors and compare the performance of the inhibitors in the tortilla formulation. A standard tortilla formulation was prepared for each test group. Approximately 500 grams of dry material were used to prepare 3 tortillas per test group. Mold inhibitors were added to the dough. The inhibitor was thoroughly mixed into the formulation. The percentage usage based on the mass of flour is outlined in Table 2 (same salts and percentages as used in previous test with bread). Three of the mold inhibitors were made with acetic and propionic acid produced from fermentation using a mixed consortium of microorganisms or microbiome using the methods disclosed herein. The natural calcium propionate (BV CP) contained over 99 wt. % calcium propionate, and the high-efficacy clean-label acetate/propionate mold inhibitors, with one having an acetate/propionate weight ratio of 1:1 (50 wt. % acetate/50 wt. % propionate) (BV1) and the other one a weight ratio of 1:6 (14 wt. % acetate/86 wt. % propionate) (BV2). These were compared to a leading commercial clean-label mold inhibitor (CCMI) and to commercial petroleum-based calcium propionate (CCP).

After mixing the tortilla formulation ingredients with the corresponding mold inhibitor, samples of rounded dough were placed onto parchment paper and allowed to ferment for 20 minutes at 82±2° F. and 85% relative humidity. The dough was then pressed into a tortilla form, cooked, cooled, and packaged. This procedure was repeated for each test group. Chemical testing included evaluation of water activity and pH, which is reported in Table 5. One replicate of dough for each group was tested prior to baking. Water activity was measured using a water activity meter. Measurements for pH were made using a pH meter. The product odor (similar to baseline or off odor development), color, and general appearance were evaluated daily. Each sample was opened and closed daily, until visible mold appeared.

TABLE 5

| Sample ID | Water Activity | pH |
|---|---|---|
| Control | 0.9558 | 6.77 |
| CCP | 0.9502 | 6.7 |
| BV1 | 0.9606 | 6.77 |
| BV1 | 0.9615 | 6.98 |
| BV2 | 0.9572 | 6.79 |
| BV2 | 0.9641 | 7.00 |
| CCMI | 0.9633 | 6.9 |
| CCMI | 0.9591 | 6.62 |
| BV CP | 0.9612 | 6.87 |
| BV CP | 0.9548 | 6.98 |

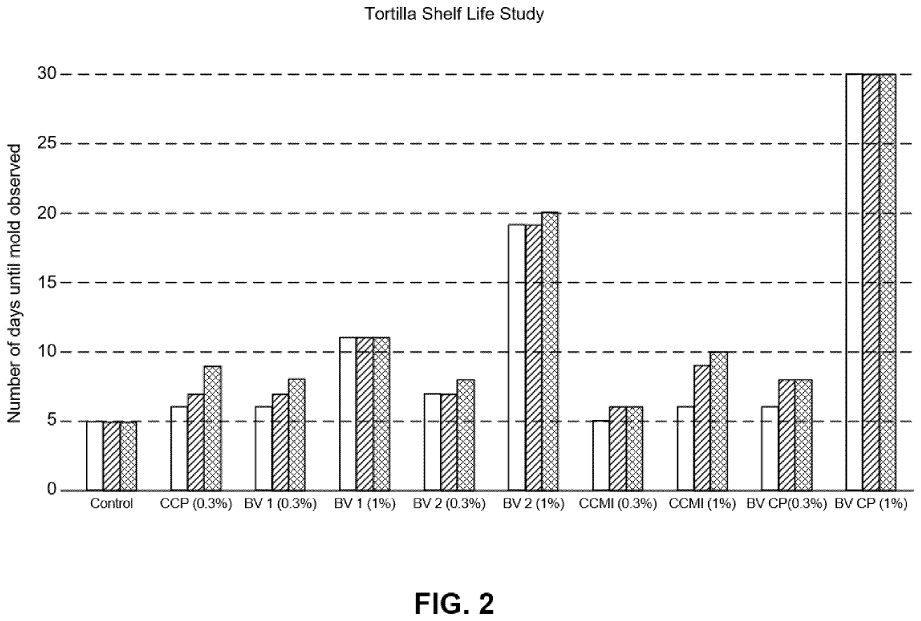

The results for the tortilla shelf-life study comparing the preservative/mold inhibitors treatments are graphically shown in FIG. 2. Three tortilla samples were produced using each of the inhibitor treatments, and the number of days until mold was observed was recorded for each sample. The samples were stored at ambient temperature from 68° F. to 73° F. over the course of the study. The dough pH results were from 6.62 to 7. The water activities for the dough were between 0.9548 and 0.9633. As mentioned, these results are presented in table 5. The control (no additive) developed visible mold on day 5. The treatment with the leading commercial clean-label mold inhibitor (CCMI), at an inclusion rate of 0.3 wt. %, developed mold on days 5 and 6, similar to the control. The treatment with the commercial petroleum-based calcium propionate (CCP) and the high-efficacy clean label acetate/propionate 1:1 (BV1) at 0.3 wt. % inclusion developed visible mold on days 6, 7, and 8 or 9. The treatment with the high-efficacy clean-label acetate/propionate 1:6 (BV2) at 0.3 wt. % inclusion developed visible mold on days 7 and 8. The leading commercial clean-label mold inhibitor (CCMI) at an inclusion of 1 wt. % developed mold on days 6, 9, and 10. Natural calcium propionate (BV CP) at 0.3 wt. % developed visible mold on days 6 and 8. All replicates for the treatment with the high-efficacy clean-label acetate/propionate 1:1 (BV1) at 1 wt. % developed visible mold on day 11. The treatment with the high-efficacy clean-label acetate/propionate 1:6 (BV2) at 1 wt. % had two replicates remain free of visible mold until day 19 of the study and one replicate developed mold at day 20. The treatment with natural calcium propionate (BV CP) at 1 wt. % had two replicates develop mold on day 30 of the study. After day 30, the samples were stored under similar conditions, but the bags were no longer opened each day. The remaining replicate with natural calcium propionate (BV CP) at 1 wt. % remained mold free through day 45 but was very dehydrated making mold growth unlikely. In summary, the treatments at an inclusion of 0.3 wt. % performed similarly except for the treatment with the leading commercial clean-label mold inhibitor (CCMI), which showed its typical lower efficacy. In fact, as mentioned, at this low inclusion rate, this leading commercial clean-label product performed similarly to the no-add control. The biggest difference was observed at the inclusion rate of 1 wt. %. At this dosage level, clearly the higher propionate concentration from acetate/propionate ratio of 1:1, then 1:6 and finally natural calcium propionate showed a longer preservation as mentioned above and shown in FIG. 2. No off odors were detected over time in any of the treatments.

Example 4. Sensory profiling of breads with added preservative salts (natural calcium propionate and commercial petroleum-based calcium propionate) was performed by a trained sensory evaluation panel at North Carolina State University, Raleigh, NC. Standard, no-time dough bread recipes were used to bake breads with mold inhibitors at a 0.5 wt. % on flour inclusion level. The mold inhibitors were mixed thoroughly into the dough. Breads were manufactured in three loaf batches. Samples were stored at room temperature. Sensory analysis was conducted on days 0, 2, and 9. A certified baker prepared the breads. A control with no added mold inhibitor was one of the samples tested by panelists. The dough and breads were evaluated at 21° C. for sensory analysis. Five panelists, each with more than 100 h of previous experience with descriptive sensory analysis of foods, including breads, evaluated the breads in duplicate at each timepoint. Descriptive analysis used a 0 to 15 point universal intensity scale with the Spectrum™ method (Meilgaard et al. 1999; Drake and Civille 2003) and a previously established bread sensory language. Paper ballots were used. Analysis of data collected from training sessions confirmed that panel results were consistent, and that terms were not redundant. Each panelist evaluated each product in duplicate. The preservative/mold inhibitor salts used in the evaluation were the natural calcium propionate (BV CP), which was prepared with propionic acid produced and extracted from mixed culture or mixed consortium fermentation per the methods disclosed herein, and a commercial petroleum-based calcium propionate (CCP). The purpose of this study was to establish whether there was a difference in the aroma/flavor profiles of each of the calcium propionates. The degree of difference (DOD) was scored on a 0-to-5-point scale by the sensory panel with 0 being identical and 5 being dramatically different. Table 6 shows the results of this sensory evaluation.

TABLE 6

| | | | | | Crumb | Crumb | |
|---|---|---|---|---|---|---|---|
| Sample | Dough | Loaf | Crumb | Crumb | sweet | sour | |
| ID | Aroma | aroma | aroma | yeasty | aromatic | aromatic | DOD |
| Day 0 | | | | | | | |
| Control | sweet, | sweet, | sweet, | 3.5 | 3.2 | 0.8 | 0 |
| | fermented | browned, | yeasty | | | | |
| | yeasty | yeasty | | | | | |
| BV CP | sweet, | sweet, | sweet, | 2.8 | 2 | 1.2 | 0.5 |
| | fermented | browned, | yeasty, | | | | |
| | yeasty, | yeasty, | slight | | | | |
| | faint sour | slight sour | sour | | | | |
| | aroma | | aromatic | | | | |
| CCP | typical | typical | typical | 3.4 | 2.9 | 0.5 | 0 |
| | like | | | | | | |
| | control | | | | | | |
| Day 2 | | | | | | | |
| Control | N/A | sweet, | sweet, | 3 | 3.2 | 1 | 0 |
| | | browned, | yeasty | | | | |
| | | yeasty | | | | | |
| BV CP | N/A | browned, | sweet, | 2.2 | 2 | 1.2 | 0.6 |
| | | yeasty, | slight | | | | |
| | | slight sour | sour | | | | |
| | | | aromatic | | | | |
| CCP | N/A | typical | typical | 2.8 | 2.9 | 0.5 | 0 |
| Day 9 | | | | | | | |
| Control | N/A | sweet, | sweet, | 2.8 | 3 | 0.7 | 0 |
| | | browned | yeasty | | | | |
| BV CP | N/A | browned, | sweet | 2.3 | 2.4 | 1.1 | 0.5 |
| | | yeasty | browned, | | | | |
| | | | less sweet | | | | |
| CCP | N/A | typical | typical | 2.8 | 2.9 | 0.5 | 0 |

Sample ID: Control (no additive), BV CP (natural calcium propionate), CCP (commercial petroleum-based calcium propionate). Flavors were scored on a 0 to 15-point universal intensity scale (Spectrum method, Meilgaard et al., 1999). Aromas not listed were not detected. Bread flavors generally fall between 0 and 5 on this scale. DOD—degree of difference was scored on a 0-to-5-point scale where 0 = identical and 5 = dramatically different.

At day 0, the dough and the baked loaf and crumb aroma were evaluated. The panelists found that the no-additive control had a “sweet, fermented, yeasty” aroma in the dough and the baked loaf and crumb. The dough, loaf, and crumb with the natural calcium propionate treatment (BV CP) were also “sweet, fermented, yeasty aroma,” but with a faint sour aroma. The dough, loaf, and crumb with the commercial petroleum-based calcium propionate (CCP) had also the same “sweet, fermented yeasty aroma.” The DOD at time 0 between no-additive control and the natural calcium propionate (BV CP) treatment was 0.5 out of 5 and the commercial petroleum-based calcium propionate (CCP) was 0. The loaf aroma was then evaluated by the panel at Day 2 and was found that both the loaf and crumb were “sweet and yeasty” and the loaf was also “browned” for the control. Similar results were found for the commercial petroleum-based calcium propionate (CCP) and with the natural calcium propionate (BV CP), but the natural calcium propionate still had a slight sour note. DOD at Day 2 was found to be 0.6 out of 5 for the natural calcium propionate (BV CP) and 0 for the commercial petroleum-based calcium propionate. Finally, at day 9, the loaf and crumb for the control were described to be “sweet and browned” and “sweet and yeasty,” respectively. For the natural calcium propionate (BV CP), the loaf was found to be “browned and yeasty” and the crumb “sweet browned” but “less sweet.” Finally, the commercial petroleum-based calcium propionate (CCP) was found to be “typical” and similar to the control. The DOD compared to the control for Day 9 was still 0.5 out of 5 for the natural calcium propionate (BV CP). DOD scores below 1.5 are not considered actionable, so for all practical purposes there were no differences between the control, the natural calcium propionate (BV CP) and the commercial petroleum-based calcium propionate (CCP).

Example 5. The sensory profiling of breads with added preservative salts (see Table 7) was performed by the sensory evaluation panel at North Carolina State University, Raleigh, NC. The preservatives were commercially available petroleum-based calcium propionate (1), a natural high-efficacy clean-label acetate/propionate preservative with an acetate/propionate ratio of 1:6 as described previously (2), and two commercial clean-label mold inhibitors from two leading brands in the market, which were manufactured using conventional means (4-7). The color of the latter two salts was different. They had a beige color compared to white for the commercial petroleum-based calcium propionate and the natural high-efficacy clean-label acetate/propionate. Standard no-time dough bread recipes were used to bake breads with an inclusion levels on flour as specified in Table 7. The reason why it was decided to run the commercial clean-label mold inhibitors at both 0.5% and 1% inclusion rates is because of their lower efficacy, which makes their typical bread inclusion rates at around 1% on flour. The mold inhibitors were mixed thoroughly into the dough. Breads were manufactured in triplicate (three loaf batches). Samples were stored at room temperature. Sensory analysis was completed at days 1 and 7. Seven breads were made using coded ingredients provided. A certified baker prepared the breads. A control with no added preservative was one of the 7 samples and this sample was labeled as a control to panelists.

TABLE 7

| Preservative salts acting as mold inhibitor | Code | % on flour |
|---|---|---|
| Control (no added preservative) | 1 | 0 |
| Commercial Petroleum-based calcium propionate | 2 | 0.5 |
| (CCP) | | |
| High-efficacy clean-label acetate/propionate 1:6 | 3 | 0.5 |
| (BV2) | | |
| Commercial clean-label mold inhibitor 1 | 4 | 0.5 |
| Commercial clean-label mold inhibitor 2 | 5 | 0.5 |
| Commercial clean-label mold inhibitor 1 | 6 | 1 |
| Commercial clean-label mold inhibitor 2 | 7 | 1 |

### Descriptive Sensory Analysis

The dough and breads were evaluated at 21° C. for sensory analysis with spring water and unsalted crackers for palate cleansing. Six panelists, each with more than 100 h of previous experience with descriptive sensory analysis of foods, including breads, evaluated the breads in duplicate at each timepoint. Descriptive analysis used a 0 to 15 point universal intensity scale with the Spectrum™ method (Meilgaard and others 1999; Drake and Civille 2003) and a previously established bread sensory language. Paper ballots were used. Analysis of data collected from training sessions confirmed that panel results were consistent and that terms were not redundant, consistent with previous use of the developed language (Drake and others 2001; Drake and others 2005). Each panelist evaluated each product in duplicate.

### Statistical Analysis

Data were analyzed by a general linear model analysis of variance with Fisher's least significant difference (LSD) as a post hoc test (SAS version 9.1, Cary, NC).

TABLE 8
Appearance and hand texture - Trained panel descriptive profiles of breads at days 1 and 7

| | | | crumb | | | | | | |
|---|---|---|---|---|---|---|---|---|---|
| | crust | crumb | cell | | | | hand | | |
| | color | color | size | | | hand | side | | hand |
| | (light | (light | (small | crumb | | side | crust | hand | crumb |
| | to | to | to | cell | | crust | rate of | crumb | rate of |
| trt | dark) | dark) | large) | uniformity | porosity | springiness | recovery | springiness | recovery |
| 1 D 1 | 12.5a | 3.0a | 10.2b | 7.7d | 11.0b | 14.1a | 12.4b | 12.9a | 11.4bc |
| 1 D 7 | 11.6b | 3.1a | 11.3a | 6.5e | 11.8a | 12.2b | 10.3c | 12.0b | 10.8d |
| 2 D 1 | 10.5cd | 3.0a | 6.9f | 9.4b | 11.7a | 14.1a | 11.9b | 12.3ab | 11.4bc |
| 2 D 7 | 10.4d | 3.0a | 6.7f | 10.1a | 11.0b | 12.6b | 10.6c | 12.4ab | 10.3e |
| 3 D 1 | 10.1d | 3.1a | 7.2f | 9.7a | 10.9b | 14.2a | 12.3b | 12.6ab | 12.1b |
| 3 D 7 | 11.3bc | 3.1a | 6.4f | 10.6a | 10.3b | 11.7c | 10.2 | 12.1b | 11.5c |
| 4 D 1 | 10.7c | 3.0a | 10.5b | 9.5ab | 12.3a | 14.0a | 12.0b | 12.5ab | 11.8bc |
| 4 D 7 | 12.3a | 3.0a | 10.0c | 9.1b | 12.2a | 11.6c | 11.0b | 11.3b | 11.4c |
| 5 D 1 | 10.6cd | 3.1a | 10.0c | 8.5c | 11.5a | 13.5ab | 12.1b | 12.3ab | 12.0b |
| 5 D 7 | 10.8c | 3.1a | 8.9d | 8.7bc | 11.5a | 12.9b | 11.8b | 12.5ab | 11.8bc |
| 6 D 1 | 10.7c | 3.1a | 8.6d | 9.1b | 10.7b | 13.9a | 11.8b | 12.3ab | 11.9b |
| 6 D 7 | 11.0c | 3.1a | 8.2e | 9.6ab | 10.3b | 12.3b | 11.6b | 11.6b | 11.5c |
| 7 D 1 | 12.5a | 2.9a | 7.9e | 8.4c | 8.8c | 13.4ab | 13.0a | 12.9a | 12.8a |
| 7 D 7 | 12.7a | 3.0a | 8.4de | 9.5ab | 8.8c | 12.8b | 11.7b | 12.1ab | 12.3b |

TABLE 9
Oral texture - Trained panel descriptive profiles of breads at days 1 and 7 (cont.)

| | | crumb | | | crumb cohes. | | crumb |
|---|---|---|---|---|---|---|---|
| | crumb | adhesiveness | top crust | crumb | Of mass @ 4 | crumb | tooth - |
| trt | hardness | to palate | chewiness | moistness | to 7 chews | chewiness | packing |
| 1 D 1 | 3.1e | 4.2d | 10.1b | 12.1a | 13.1a | 10.0d | 2.9e |
| 1 D 7 | 3.1e | 4.1d | 9.3d | 10.7c | 12.5b | 9.3e | 3.0e |
| 2 D 1 | 4.2c | 4.4c | 10.1b | 11.4b | 13.1a | 10.8b | 4.9c |
| 2 D 7 | 4.8b | 5.4a | 9.7c | 7.9e | 12.5b | 10.9b | 5.7b |
| 3 D 1 | 4.8b | 4.6c | 10.2b | 10.6cd | 13.0a | 11.8a | 4.5cd |
| 3 D 7 | 5.6a | 4.8bc | 10.6a | 7.8e | 13.2a | 11.1b | 6.4a |
| 4 D 1 | 3.3de | 5.0b | 10.1b | 11.1bc | 12.5b | 10.8b | 4.8c |
| 4 D 7 | 3.5d | 4.7bc | 10.8a | 10.0d | 12.0c | 10.4c | 4.8c |
| 5 D 1 | 3.5d | 4.6c | 10.1b | 10.4d | 12.9a | 10.3cd | 4.5cd |
| 5 D 7 | 3.7d | 5.5a | 9.7c | 10.2d | 12.5b | 10.6bc | 5.4bc |
| 6 D 1 | 3.5d | 4.5c | 10.1b | 11.6b | 13.1a | 10.6bc | 4.8c |
| 6 D 7 | 4.6b | 4.8bc | 9.9b | 10.3d | 12.4b | 10.5c | 5.0c |
| 7 D 1 | 3.6d | 3.9d | 10.0bc | 11.1bc | 13.0a | 9.3e | 4.3d |
| 7 D 7 | 3.9c | 4.7bc | 9.5d | 10.3d | 12.9a | 9.2e | 4.2d |

TABLE 10
Crust flavor -Trained panel descriptive profiles of breads at days 1 and 7 (cont.).

| | sw | white | | | | | | | | | |
|---|---|---|---|---|---|---|---|---|---|---|---|
| | aromatic | flour | sour | burned/ | astr | | | | | | aft |
| trt | caramelized | paste | aromatic | tstd | mf | sweet | sour | salty | bitter | umami | int. |
| 1 D 1 | 2.3a | 1.1a | 1.0d | 1.5bc | 2.1b | 2.4bc | 1.6d | 3.0a | 1.0a | 2.3c | 2.1a |
| 1 D 7 | 2.1b | 1.1a | 0.5e | 1.4c | 2.1b | 2.3c | 1.8c | 3.0a | 1.0a | 2.3c | 2.1a |
| 2 D 1 | 2.3a | 1.1a | 1.1d | 1.4c | 2.0b | 2.6b | 1.9bc | 3.0a | 1.0a | 2.5bc | 2.1a |
| 2 D 7 | 2.0bc | 1.1a | 1.3cd | 1.2d | 2.0b | 2.5b | 1.9bc | 3.0a | 0.9a | 2.5bc | 2.0a |
| 3 D 1 | 2.1b | 1.1a | 1.4c | 1.1d | 2.0b | 3.0a | 1.9bc | 3.0a | 1.0a | 2.5bc | 2.1a |
| 3 D 7 | 2.0bc | 1.1a | 1.5c | 1.4c | 2.0b | 3.0a | 2.0b | 3.0a | 1.0a | 2.7b | 2.1a |
| 4 D 1 | 1.9c | 1.0a | 2.1a | 1.8ab | 2.1b | 1.9d | 2.4a | 3.0a | 1.0a | 2.6b | 2.1a |
| 4 D 7 | 1.9c | 1.1a | 2.2a | 2.1a | 2.2b | 2.0d | 2.4a | 3.0a | 1.0a | 2.7b | 2.1a |
| 5 D 1 | 2.4a | 1.1a | 2.1a | 1.6b | 2.1b | 1.9d | 2.4a | 3.0a | 1.0a | 2.7b | 2.1a |
| 5 D 7 | 2.4a | 1.1a | 2.0a | 1.7b | 2.0b | 2.0d | 2.4a | 3.0a | 1.1a | 2.7b | 2.0a |
| 6 D 1 | 1.2d | 1.1a | 2.0a | 1.6b | 2.1b | 2.1d | 2.2b | 3.0a | 1.0a | 2.7b | 2.1a |
| 6 D 7 | 1.1d | 1.1a | 2.0a | 1.7b | 2.2b | 1.9d | 2.3ab | 3.0a | 1.1a | 2.8ab | 2.1a |
| 7 D 1 | 1.1d | 1.0a | 1.7b | 1.9a | 2.5a | 2.1d | 2.1b | 3.0a | 1.0a | 2.9a | 2.1a |
| 7 D 7 | 1.1d | 1.1a | 1.2d | 2.0a | 2.6a | 2.1d | 2.1b | 3.0a | 1.0a | 3.0a | 2.1a |

TABLE 11
Crumb flavor - Trained panel descriptive profiles of breads at days 1 and 7 (cont).

| | sw | | white | | | | | | | | |
|---|---|---|---|---|---|---|---|---|---|---|---|
| | aromatic | sour | flour | yeasty/ | | astr | | | | | aft |
| trt | caramelized | aromatic | paste | fermented | chemical | mf | sweet | sour | salty | umami | int. |
| 1 D 1 | 2.8a | 1.1b | 1.3ab | 2.1b | ND | 1.6a | 3.1a | 0.6c | 3.1a | 2.9a | 2.1a |
| 1 D 7 | 3.0a | 1.1b | 1.3ab | 2.0b | ND | 1.6a | 3.1a | 0.6c | 3.0a | 3.0a | 2.1a |
| 2 D 1 | 2.5b | 1.6a | 1.2b | 2.1b | ND | 1.8a | 3.1a | 0.6c | 3.1a | 2.9a | 2.1a |
| 2 D 7 | 2.0c | 0.5c | 1.4a | 2.0b | ND | 1.7a | 3.1a | 0.6c | 3.1a | 2.8a | 2.1a |
| 3 D 1 | 2.5b | 1.2b | 1.4a | 2.5a | ND | 1.5a | 3.1a | 0.9b | 3.1a | 3.1a | 2.0a |
| 3 D 7 | 2.1c | 1.0b | 1.1b | 2.4a | ND | 1.6a | 3.1a | 0.8bc | 3.1a | 3.1a | 2.1a |
| 4 D 1 | 2.1c | 0.8bc | 1.2b | 2.1b | ND | 1.8a | 2.2c | 1.1b | 3.1a | 3.0a | 2.0a |
| 4 D 7 | 1.6d | 0.8bc | 1.1b | 2.0b | ND | 1.6a | 2.1c | 1.0b | 3.1a | 3.1a | 2.1a |
| 5 D 1 | 1.5d | 1.5a | 1.0b | 2.2ab | ND | 1.7a | 2.1c | 1.8a | 3.1a | 3.1a | 2.1a |
| 5 D 7 | 1.5d | 1.0b | 1.1b | 2.2ab | ND | 1.7a | 2.0c | 1.8a | 3.1a | 3.1a | 2.1a |
| 6 D 1 | 1.5d | 0.6c | 1.1b | 2.2ab | 2.0a | 1.6a | 2.2c | 1.1b | 3.1a | 3.1a | 2.1a |
| 6 D 7 | 1.6d | 0.6c | 1.1b | 2.0b | 2.1a | 1.6a | 2.1c | 1.1b | 3.1a | 3.0a | 2.1a |
| 7 D 1 | 2.1c | 1.0b | 1.4a | 2.0b | ND | 1.6a | 2.7b | 1.6a | 3.1a | 3.1a | 2.1a |
| 7 D 7 | 1.9c | 0.6c | 1.5a | 1.7c | ND | 1.6a | 3.0a | 1.7a | 3.1a | 3.0a | 2.1a |

For Tables 8 through 11, “trt” number refers to treatment, as per Table 7, D refers to day 1 or day 7 after baking. Flavors were scored on a 0 to 15-point universal intensity scale (Spectrum method, Meilgaard et al., 1999). Flavors not listed were not detected. Bread flavors generally fall between 0 and 5 on this scale. Visual and texture attributes were scored on a 0 to 15 points product specific intensity scale. ND—not detected. Means in a column followed by a different letter are different (p<0.05).

TABLE 12
Factor loadings

| | F1 | F2 | F3 |
|---|---|---|---|
| crust color | 0.457 | −0.520 | −0.489 |
| crumb cell size | 0.312 | −0.561 | 0.523 |
| crumb cell uniformity | −0.071 | 0.833 | −0.270 |
| porosity | −0.344 | 0.071 | 0.800 |
| hand side crust springiness | −0.105 | −0.367 | 0.377 |
| hand side crust rate of recovery | 0.405 | −0.352 | 0.226 |
| hand crumb springiness | −0.225 | −0.367 | 0.042 |
| hand crumb rate of recovery | 0.615 | −0.134 | −0.019 |
| crumb hardness | −0.404 | 0.712 | −0.486 |
| crumb adhesiveness to palate | −0.026 | 0.762 | 0.186 |
| crumb moistness | 0.263 | −0.704 | 0.395 |
| crumb chewiness | −0.527 | 0.656 | 0.345 |
| crumb tooth - packing* | −0.050 | 0.950 | −0.101 |
| sw aromatic caramelized | −0.599 | −0.019 | 0.577 |
| sour aromatic | 0.618 | 0.574 | 0.427 |
| burned / tstd | 0.905 | −0.027 | −0.017 |
| astr mf | 0.708 | −0.295 | −0.589 |
| sweet | −0.839 | 0.107 | −0.334 |
| sour | 0.710 | 0.524 | 0.420 |
| umami | 0.740 | 0.478 | −0.276 |
| crumb sw aromatic caramelized | −0.653 | −0.665 | −0.198 |
| crumb sour aromatic | −0.321 | −0.349 | 0.395 |
| crumb yeasty / fermented | −0.572 | 0.289 | 0.393 |
| crumb sweet | −0.663 | −0.326 | −0.613 |
| crumb sour | 0.784 | 0.153 | 0.153 |

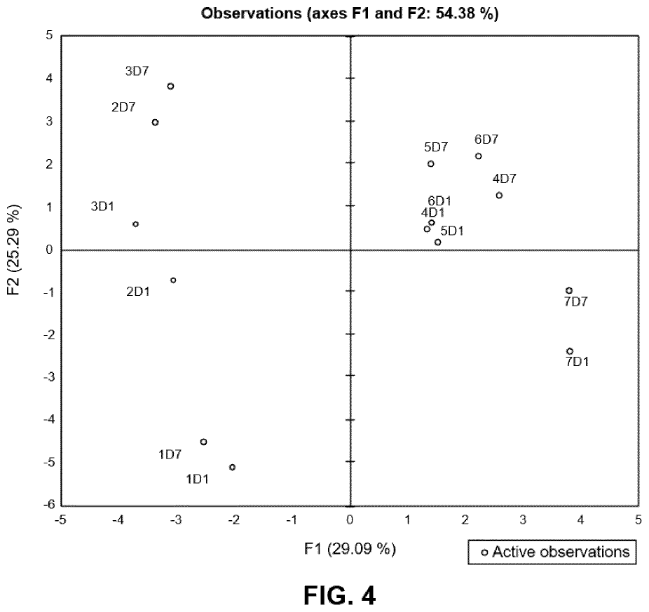

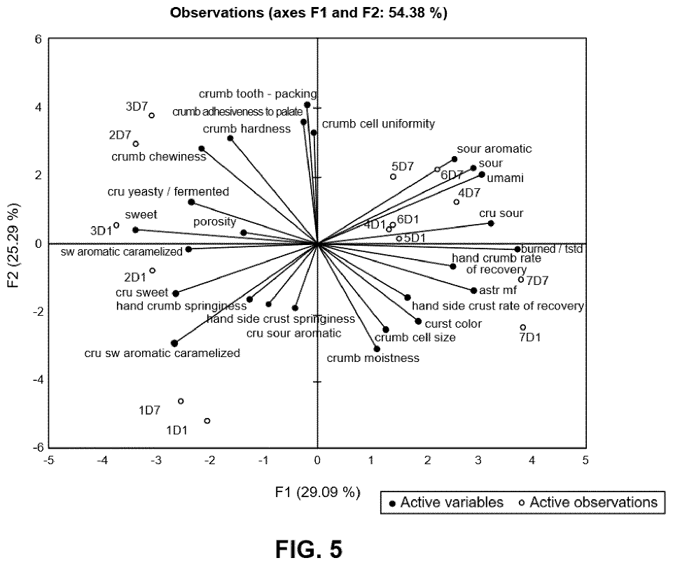

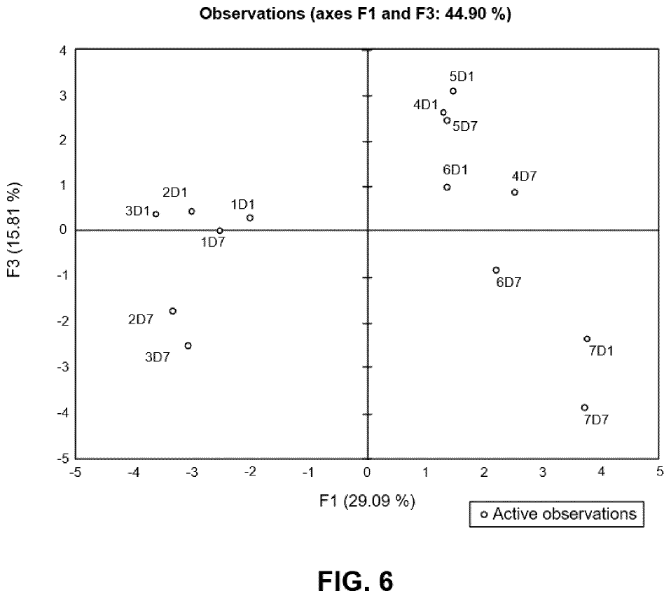

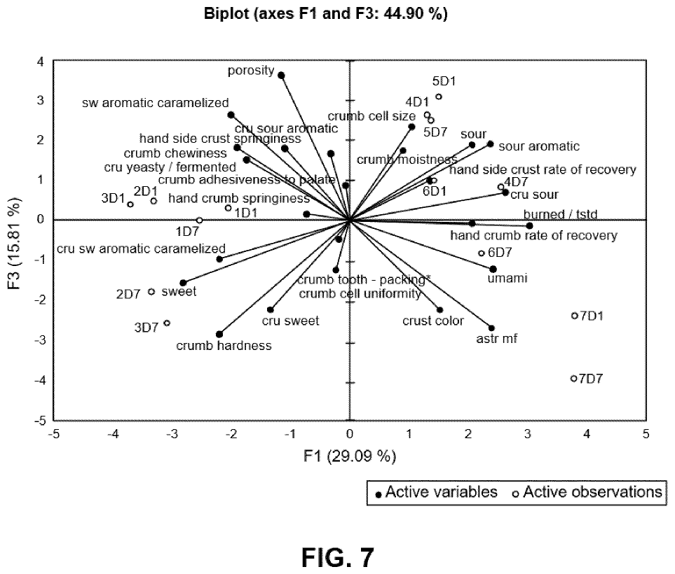

FIGS. 4-7 illustrate the Principal Component Analysis (PCA) biplots of the sensory means of the different breads for factor loading F2 vs F1 and F3 vs F1. In the figures, a clear clustering to the left of the treatments 2 and 3 (i.e., Commercial Petroleum-based calcium propionate and high-efficacy clean-label acetate/propionate 1:6) is observed for both 1 and 7 days after baking, while treatments 4 through 7 for the commercial-clean label mold-inhibitors at different inclusion rates cluster to the right. This shows that although treatment 3 (high-efficacy clean-label acetate/propionate 1:6) contains acetate and not just propionate, the sensory (flavor, appearance, and texture) attributes do not differ much from that of the traditional commercial petroleum-based calcium propionate. On the other hand, some difference can be realized for the commercial clean-label mold inhibitors when compared to commercial petroleum-based calcium propionate. In addition, the differences between day 1 and 7, for the commercial clean-label mold inhibitors are also more drastic than that observed for commercial petroleum-based calcium propionate and high-efficacy clean-label acetate/propionate.

Example 6. In another example, a comparative shelf-life and sensory study using pup loaves was performed by an ISO/IEC 17025:2017 certified laboratory, Great Plains Analytical Laboratory, Kansas City, MO. A total of seven test groups of white bread in a pup loaf formulation were prepared. The test groups were made up of two levels of commercial calcium propionate (CCP), four different inclusion levels of the high-efficacy clean-label acetate/propionate mold inhibitors, with one having an acetate/propionate weight ratio of 1:7 (13 wt. % acetate/87 wt. % propionate) (BV3) and one level of a commercial calcium propionate/sorbic acid blend. Test groups were stored at ambient temperature for between 7 and 15 days to determine the overall efficacy of the inhibitors and compare the performance of the inhibitors in the bread formulation. Sensory analysis was performed examining the flavor and crumb texture for all samples. The studies were conducted concurrently.

A standard white bread dough formulation was prepared for each test group. Three pup loaves were prepared for each test group for the visual examination portion of the study. Mold inhibitors were added to the dry ingredients prior to mixing. The inhibitor was thoroughly mixed into the formulation. The percentage usage of mold inhibitor is outlined in Table 13. After mixing, the dough was divided into bread pans and allowed to ferment for three hours at 86±1° F. and 60% relative humidity. The dough was run through the molder, panned, and then proofed for 55 minutes at 109±1° F. and 90% humidity. The bread was baked for 15 minutes at 425±1° F. Internal temperatures were recorded upon exiting the oven and prior to packaging. The procedure was repeated for each test group for a total of 18 loaves of bread for the shelf-life portion of the study.

TABLE 13
Usage of mold inhibitor for each test group.

| Test Group | Mold Inhibitor (wt. %) | |
|---|---|---|
| 1 (CCP) | 0.5 | wt. % |
| 2 (CCP) | 0.8 | wt. % |
| 3 (BV3 Level A) | 0.5 | wt. % |
| 4 (BV3 Level B) | 0.6 | wt. % |
| 5 (BV3 Level C) | 0.7 | wt. % |
| 6 (BV3 Level D) | 0.8 | wt. % |
| 7 CCP/Sorbic | 0.28/0.11 | wt. % |

Chemical testing included the evaluation of pH on the dough out of the mixer (Table 15). Measurement for pH was made using a pH meter. A bake score, height, and volume were recorded for each test group. The bake score examined the crust color, loaf symmetry, uniformity of the break and shred, crumb texture, crumb grain, crumb color, taste, and aroma. The pup loaves were evaluated for visible mold every day for the shelf-life portion of the study. A photograph of the visible mold was taken the day it was detected. The sensory analysis consisted of a quantitative descriptive analysis and a ranking performed by a trained panel. The ranking portion provided a rank from most preferred to least preferred from the individual panel members and the panel as a group. The descriptive analysis covered the crumb texture and flavor of the test groups. The sensory analysis was performed on Days 3, 6, 9, 12, and 15. No sensory analysis was performed on bread that had visible mold.

The temperature of the loaves out of the oven and prior to packaging were recorded in Table 14. Dough was removed from the mixing bowl and then analyzed for pH. The results of this analysis are found in Table 15.

TABLE 14
Bread Temperatures Out of the Oven and Before Packaging

| | Internal Temperature | Internal Temperature |
|---|---|---|
| Test Group | Out of Oven (° F.) | Before Packaging (° F.) |
| 1 | 202.1 | 67.6 |
| 2 | 202.4 | 67.9 |
| 3 | 202.7 | 67.6 |
| 4 | 202.8 | 68.1 |
| 5 | 203.1 | 68.6 |
| 6 | 201.8 | 68.4 |
| 7 | 202. | 68.9 |

TABLE 15
The dough pH values for each test group
taken after exiting the mixer.

| Test Group | Dough pH |
|---|---|
| 1 | 5.34 |
| 2 | 5.53 |
| 3 | 5.31 |
| 4 | 5.33 |
| 5 | 5.22 |
| 6 | 5.21 |
| 7 | 5.16 |

Immediately out of the oven, volumes were taken using a volumeter (Table 16). Groups 1, 3, and 4 had very good volumes. Groups 2, 5, and 6 had excellent volumes of greater than 800 cm³. Group 7 had very poor volume. The two highest percentages of BV3 produced improved loaf volumes compared to the two lower percentages of BV3. The 0.8% BV3 loaf had the best volume out of all of the test groups.

The bake score was completed the day after baking (Table 17). Group 1 had a dull crust color and the darkest crust color compared to all the other test groups. The loaf had good symmetry and a slightly uneven break and shred. The texture was slightly harsh with a close grain and creamy crumb color. Test group 2 had a dull crust color with good symmetry and uniformity. The texture was slightly harsh with a uniform grain. The crumb color was creamy with typical aroma and taste. Group 3 had very slightly light crust color and good symmetry and uniformity. The texture was slightly rough with a somewhat open grain, creamy crumb color and typical aroma and taste. Group 4 had slightly light crust color with good symmetry and a double-sided break and shred. The texture was slightly rough with a somewhat open grain and creamy crumb color. No defects in taste or aroma were detected. Group 5 had slightly light crust color with good symmetry and a wild break and shred. The texture was slightly rough with a uniform grain and creamy crumb color. Group 6 had the lightest crust color of all the test groups. The loaf had good symmetry with a uniform break and shred. The texture was slightly rough with a somewhat open grain and creamy crumb color. The aroma and taste were typical. Group 7 had rich color development, and the best crust color score out of all the test groups. The loaf was symmetrical with a short double-sided break and shred. The texture was very harsh with a very tight grain and dull crumb color.

TABLE 16
The loaf weight, volume, and specific
volume immediately out of the oven.

| Test Group | Weight (g) | Volume (cc) | Specific Volume (cc/g) |
|---|---|---|---|
| 1 | 150 | 797 | 5.313 |
| 2 | 146 | 818 | 5.603 |
| 3 | 151 | 776 | 5.139 |
| 4 | 153 | 793 | 5.183 |
| 5 | 155 | 821 | 5.297 |
| 6 | 157 | 839 | 5.344 |
| 7 | 157 | 588 | 3.745 |

TABLE 17
The bake score out of 100 points for each test group.

| Attribute | Group 1 | Group 2 | Group 3 | Group 4 | Group 5 | Group 6 | Group 7 |
|---|---|---|---|---|---|---|---|
| Crust (10) | 8 | 7 | 7 | 7 | 7 | 6 | 9 |
| Symmetry (10) | 8 | 8 | 8 | 8 | 8 | 8 | 8 |
| Uniformity (10) | 7 | 8 | 8 | 7 | 7 | 8 | 6 |
| Texture (20) | 16 | 16 | 16 | 16 | 16 | 16 | 14 |
| Grain (20) | 15 | 17 | 16 | 16 | 16 | 16 | 14 |
| Crumb Color (10) | 6 | 6 | 6 | 6 | 6 | 6 | 5 |
| Aroma (10) | 10 | 10 | 10 | 10 | 10 | 10 | 6 |
| Taste (10) | 10 | 10 | 10 | 10 | 10 | 10 | 6 |
| Total Score (100) | 80 | 82 | 81 | 80 | 80 | 80 | 68 |

The loaves were observed for mold each day. Results of the observations indicating the first day on which mold was visibly observed on the exterior of the pup loaf are shown in Table 18.

TABLE 18
Day of visible mold observation.

| Test Group Number | Day of Visible Mold |
|---|---|
| 1 | 7 |
| 2 | 9 |
| 3 | 7 |
| 4 | 7 |
| 5 | 7 |
| 6 | 8 |
| 7 | 15 |

The sensory profiles were constructed by the trained panel and a list of descriptors was generated. Sensory analysis focused on the texture and flavor attributes of the bread samples. The texture attributes were moistness, chewiness, hardness, gumminess, brittleness, and overall crumb texture. The flavor attributes were off-flavor, aftertaste, stale taste, earthy flavor, sour flavor, and musty aroma of the bread. The different descriptors were quantified using an intensity scale. The order of presentation was randomized between panel members. Water was provided for rinsing between samples.

There were statistically significant differences (p<0.05) found between the flavor and texture attributes examined on both day 3 and day 6. Test groups 1, 2, 3, and 6 were all found to have little to no off flavor, aftertaste, stale taste, earthy flavor, sour flavor, or musty aroma. The panel detected an aftertaste in test groups 4, 5, and 7. The aftertaste in groups 4 and 5 was described as slightly sour. The aftertaste was described as bitter in test group 7. The panel detected a chemical off flavor, slight musty aroma, earthy flavor, and stale taste in test group 7. The high stale taste score is likely from the panel perceiving that this sample was quite dry during sensory analysis on day 3.

For texture attributes, test groups 6 and 4 were moist, had a very soft, smooth, elastic crumb texture, a very tender bite, and a slightly gummy mouthfeel. Test group 7 had a firm, very smooth, elastic crumb texture, but was slightly tough and very dry. The panel's score indicates that the grain on test group 7 loaves is very tight, which would lead to a very smooth texture in the mouth. Test groups 2 and 3 were similar with a slightly moist, slightly elastic, soft, smooth crumb texture. The bite was tender with a slightly gummy mouthfeel. Test group 5 was very moist with a smooth, very soft, elastic crumb texture. The bite was very tender with a gummy mouthfeel. Test group 1 was dry with a smooth, slightly crumbly, firm crumb texture. The bite was tender with a slightly gummy mouthfeel.

For flavor attributes on day 6, the panel described test group 7 as having a significant chemical off flavor. The bitter aftertaste, stale taste, earthy flavor, and musty aroma were still present on day 6. Test group 1 had a slight chemical off flavor, slight musty aroma, stale taste, and bitter aftertaste. Test group 2 was described as having a slight chemical off flavor, stale taste, slight sour flavor, earthy flavor, and slight bitter aftertaste. Test group 3 developed a slight earthy flavor, slight stale taste, and slight bitter aftertaste on day 6. Test groups 4, 5, and 6 were described as having little to no off flavor, aftertaste, stale taste, earthy flavor, sour flavor, or musty aroma.

On day 6, the panel noted that test groups 1, 2, and 7 were very dry with a firm, coarse, and crumbly crumb texture. The bite was tough with a slightly mealy mouthfeel. Test group 3 was dry with a slightly coarse, crumbly, slightly firm crumb texture. The bite was slightly tough with a mealy mouthfeel. Test group 5 was moist with a smooth, very soft, elastic crumb texture. The bite was tender with a slightly gummy mouthfeel. Test group 4 was moist with a slightly coarse, slightly soft, elastic crumb texture. The bite was slightly tender with a slightly gummy mouthfeel. Test group 6 was moist with a smooth, elastic, very soft crumb texture. The bite was very tender with a slightly gummy mouthfeel.

The panel consistently ranked test group 6 as the most preferred on both analysis days (Tables 19 and 20). Panel comments were that group 6 loaves were soft and had little to no aftertaste or off flavor. On average, test group 4 came in as the next most preferred, also with little to no off flavor and aftertaste. Test group 7 was the least preferred on both analysis days. Group 7 samples were described as dry with off flavors and a bitter aftertaste.

Sensory analysis continued as scheduled for group seven on days 9 and 12. The stale taste and sour flavor increased on days 9 and 12 with the other flavor attributes remaining consistent.

TABLE 19
Panel preference rankings of the test groups
individually and as a group on day 3.

| Panelist | 230 | 231 | 232 | 233 | 234 | 235 | Group |
|---|---|---|---|---|---|---|---|
| Most Preferred | 6 | 6 | 6 | 6 | 6 | 6 | 6 |
| | 4 | 4 | 3 | 3 | 2 | 5 | 4 |
| | 2 | 3 | 4 | 1 | 5 | 3 | 3 |
| | 5 | 1 | 2 | 4 | 4 | 4 | 5 |
| | 3 | 5 | 5 | 5 | 3 | 2 | 2 |
| | 1 | 2 | 1 | 2 | 1 | 1 | 1 |
| Least Preferred | 7 | 7 | 7 | 7 | 7 | 7 | 7 |

TABLE 20
Panel preference rankings of the test groups
individually and as a group on day 6.

| Panelist | 230 | 231 | 232 | 233 | 234 | 235 | Group |
|---|---|---|---|---|---|---|---|
| Most Preferred | 6 | 6 | 6 | 6 | 6 | 6 | 6 |
| | 4 | 4 | 4 | 4 | 5 | 5 | 4 |
| | 3 | 5 | 5 | 3 | 3 | 4 | 5 |
| | 5 | 3 | 3 | 1 | 4 | 3 | 3 |
| | 2 | 1 | 1 | 5 | 2 | 2 | 1 |
| | 1 | 2 | 2 | 2 | 1 | 1 | 2 |
| Least Preferred | 7 | 7 | 7 | 7 | 7 | 7 | 7 |

The calcium propionate/sorbic acid blend consistently had the highest off flavor and aftertaste scores across all test groups and sensory analysis intervals. It had very poor volume and a smooth but very firm, dry crumb. On mold inhibition, it performed the best compared to the other test groups, but this inhibitor may be designed to have dough improvers in the formulation to offset the loss in volume.

### REFERENCES

Agler, M. T. et al., “Chain elongation with reactor microbiomes: Upgrading dilute ethanol to medium-chain carboxylates,” Energy Environ. Sci., 5 (8) (2012), 8189-8192.
Barr, R. S. and Newsham, D. M. T. “Freezing temperatures of water, alkanoic acids and their mixtures,” The Chemical Engineering Journal, 33 (1986) 79-86.
den Besten, G. et al “The role of short-chain fatty acids in the interplay between diet, gut microbiota, and host energy metabolism,” Journal of Lipid Research, 54 (2013), 2325-2340.
Doores, S. “Organic Acids,” In: “Antimicrobials in food,” Ed: Davidson, P. M., Sofos, J. N., Branen A. L., CRC Press, Boca Raton, F L, 2005, pp 91.
Drake, M. A. and Civille, G. V. “Flavor lexicons,” Comprehensive Reviews in Food Science and Food Safety, 2:1 (2003), 33-40.
Holtzapple et al. “Microbial communities for valorizing biomass using the carboxylate platform to produce volatile fatty acids: A review,” Biores. Technol. 344 (2022) 126253.
Meilgaard, M. C. et al. “The Spectrum™ Descriptive Analysis Method,” In: “Sensory evaluation techniques: Third Edition,” Ed: Meilgaard, M. C., Carr, B. T., Civille, G. V., CRC Press, Boca Raton, F L, 1999, pp. 175.
Nachay, K. “Food Preservation in a Clean Label Era,” Food Technology Magazine, 74:1 (2020), (https://www.ift.org/news-and-publications/food-technology-magazine/issues/2020/january/columns/food-preservation-in-a-clean-label-era).
Ríos-Covián, D. et al. “Intestinal Short Chain Fatty Acids and their Link with Diet and Human Health,” Front Microbiol. (2016) 7, 185 (https://pubmed.ncbi.nlm.nih.gov/26925050/).
Stopforth, J. D. et al. “Sorbic acid and sorbates,” In: “Antimicrobials in food”, Ed: Davidson, P. M., Sofos, J. N., Branen A. L., CRC Press, Boca Raton, F L, 2005, pp 49.
Weimer, P. J. et al. “Lessons from the cow: What the ruminant animal can teach us about consolidated bioprocessing of cellulosic biomass,” Biores. Technol., 100 (2009), 5323-5331. Younes, M. et al. “Safety evaluation of buffered vinegar as a food additive,” EFSA Journal 20 (2022) 7351, 21 pp (https://doi.org/10.2903/j.efsa.2022.7351).

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method for producing a preservative salt, the method comprising:
feeding a biodegradable feedstock to a fermentation to produce a fermentation effluent comprising a carboxylic acid mixture;
recovering the carboxylic acid mixture from the fermentation effluent;
separating an acid fraction from the carboxylic acid mixture, the acid fraction comprising acetic acid and propionic acid; and
reacting the acid fraction with a base to produce the preservative salt, wherein the preservative salt comprises acetic acid salt and propionic acid salt.

2. The method of claim 1, wherein the biodegradable feedstock comprises a food industry byproduct, a food industry waste, an agricultural industry byproduct, an agricultural industry waste, or a mixture of any two or more thereof.

3. The method of claim 1, wherein the separating comprises distillation or freeze crystallization.

4. The method of claim 1, wherein the base comprises sodium hydroxide, potassium hydroxide, calcium hydroxide, sodium carbonate, potassium carbonate, calcium carbonate, sodium bicarbonate, potassium bicarbonate, calcium bicarbonate, or a mixture of any two or more thereof.

5. The method of claim 1 further comprising adding water to the acid fraction or to the acetic acid salt and the propionic acid salt.

6. The method of claim 5 further comprising preparing a salt solution from the acetic acid salt and propionic acid salt and filtering the salt solution.

7. The method of claim 1 further comprising drying the acetic acid and the propionic acid salt with a spray dryer or a drum dryer.

8. The method of claim 1 further comprising crystallizing, recovering, and drying the acetic acid and the propionic acid salt to produce a powder.

9. The method of claim 7 further comprising grinding and sifting the acetic acid and the propionic acid salt to achieve a desired particle size.

10. The method of claim 1, wherein the preservative salt comprises from about 60 wt. % to about 80 wt. % acetate and propionate on a dry basis.

11. The method of claim 10 wherein the acetate and propionate portion of the preservative salt comprises from about 50 wt. % to about 99.9 wt. % propionate on a dry basis.

12. A preservative salt prepared by the method of claim 1.

13. The method of claim 1, wherein the preservative salt comprises calcium propionate.

14. A preservative salt manufactured by a method comprising:
fermenting a biodegradable feedstock to produce a fermentation effluent comprising a carboxylic acid mixture comprising carboxylic acids and carboxylic acid salts;
recovering the carboxylic acid mixture from the fermentation effluent in a carboxylic acid recovery system;
separating an acid fraction from the carboxylic acid mixture, the acid fraction comprising acetic acid and propionic acid; and
reacting the acetic acid and propionic acid of the acid fraction with a base to neutralize and produce the preservative salt.

15. The preservative salt in claim 14, wherein the preservative salt comprises from about 60 wt. % to about 80 wt. % acetate and propionate on a dry basis or from about 70 wt. % to about 80 wt. % acetate and propionate on a dry basis.

16. The preservative salt in claim 15, wherein a combined mass of acetate and propionate in the preservative salt comprises from 50 wt. % to about 99.9 wt. % propionate on a dry basis or from about 80 wt. % to about 90 wt. % propionate on a dry basis.

17. A food product comprising the preservative salt of claim 14, wherein the preservative salt is substantially free of petroleum-based propionate salts.

18. The food product of claim 17, wherein the preservative salt is a natural or clean-label preservative in the food product.

19. A baked composition comprising the preservative salt of claim 14.

20. The baked composition of claim 19, wherein the preservative salt comprises calcium propionate.

21. The baked composition of claim 19, wherein the baked composition maintains a degree of difference in sensory testing of less than 1.5 after 9 days of aging when compared to a second baked composition comprising calcium propionate prepared by reaction of a calcium salt with a petroleum-derived propionic acid.

22. The baked composition of claim 21, wherein the baked composition comprises a flour, and the calcium propionate is present at about 0.3 to 1.0 wt. % based on a total dry weight of the flour in the baked composition.

23. A method for producing a baked composition, the method comprising mixing a preservative salt into a dough, wherein the preservative salt is derived from carboxylic acids produced via fermentation of a biodegradable feedstock.

24. The method of claim 23, wherein a mass of the preservative salt mixed into the dough is about 0.3 to 1.0% of a mass of flour in the dough.

25. The method of claim 1, wherein the fermentation comprises microorganisms of the genus Propionibacterium.

26. The method of claim 1, wherein the fermentation comprises a mixed consortium of microorganisms.
